(19) United States
(12) Patent Application Publication
(10) Pub. No.: US 2010/0203626 A1
(43) Pub. Date: Aug. 12, 2010

(54) SOLID STATE FERMENTATION IN MODIFIED ZYMOTIS PACKED BED BIOREACTOR

(75) Inventors: Parisa Hejazi, Tehran (IR); Seyed Abbas Shojaosadati, Tehran (IR); Zohreh Hamidi-Esfahani, Tehran (IR); Ebrahim Vasheghani-Farahani, Tehran (IR)

Correspondence Address: CHOOBIN & CHOOBIN CONSULTANCY L.L.C;Barry Choobin, Green Community, Building 3, Ground Floor, Dubai Investment Park, P.O Box 212880, Dubai, AE

(73) Assignees: TARBIAT MODARES UNIVERSITY, Tehran (IR); Industrial Development, and Renovation Organizatio, Tehran (IR)

(21) Appl. No.: 12/758,817

(22) Filed: Apr. 13, 2010

(51) Int. Cl. C12M 1/12

(52) U.S. Cl. 435/305.1

(57) ABSTRACT

The embodiments herein provide an improved Zymotis solid-state bioreactor and its operation for cultivation of microorganisms aseptically. In one embodiment, a bioreactor has two compartments containing three cooling plates. These plates have internal baffles instead of cooling tubes. The bioreactor has temperature sensors are loaded at different heights of the outer cooling plates for recoding and monitoring bed temperature during fermentation. Short space between two cooling plates with suitable material construction permits metabolic heat removal by conduction. The distance between two compartments is adjustable manually to achieve the best width. The product is extracted and harvested in the bioreactor using a trickle solvent. All of the on-line data are monitored on screen and recorded in the computer. Microorganisms are cultivated in such a manner that the bioreactor carries out all steps for cultivating microorganisms in an aseptic environment.

### CROSS-REFERENCE TO RELATED APPLICATION

This application claims the priority under 35 USC 119(e) of U.S. Provisional Application Ser. No. 61/290033, which is included by reference herein.

### SPONSORSHIP STATEMENT

The present invention is sponsored by Tarbiat Modares University and Industrial Development, and Renovation Organization of Iran (IDRO).

### BACKGROUND

1. Technical Field

The embodiment herein generally to the field of bioreactor and particularly to the solid-state fermentation in the bio reactor. The embodiment herein more particularly relates to a modified Zymotis packed bed reactor for microbial cultivation and harvesting of final product under controlled aseptic condition.

2. Description of the Related Art

Solid-state fermentation (SSF) involves the growth of any genus of microorganisms on moist solid substrates in the absence of free water. This cultivation technique has potential to be used at commercial scale for production of some microbial products such as enzymes, organic acids and the most of the secondary metabolites (Krisshna, 2005; Pandey, 2003).

One of the simplest designs of solid state fermentations (SSF) are packed bed bioreactors which have potential, particularly for those fungal fermentations in which agitation is harmful (Durand, 2003; Shojaosadati and Babaeipour 2001). Due to the absence of free-flowing water and low thermal conductivity of the solid substrates, removal of the heat produced by the growing microorganisms in these systems, can be problematic and adversely affect the microbial activity (Weber et al., 2002; Hamidi-Esfahani et al., 2004, Shojaosadati et al., 2007). The available operating variables for the control of the bed temperature and moisture in a packed bed bioreactor are inlet air temperature, air flow rate and humidity of the inlet air, moisture of the substrate at the beginning of the operation and cooling water temperature in the bioreactor wall (Lillo et al., 2001; Von Meien et al., 2004; Ashley et al., 1999).

Cooling of bed by forced aeration, due to evaporation, is the main effective way to overcome temperature and moisture gradients of the bed during the static operation (Mitchell et at., 2000a; Nagel et al., 2001). Although tray bioreactors can be used as static bioreactors, packed beds are more appropriate because the forced aeration allows one to have some control over fermentation variables through manipulation of the flow rate and the temperature of the air used in the fermentation. However, axial forced aeration results in the evaporation of water, the desiccation of a substrate and also the axial temperature and moisture gradients. Even with water saturated inlet air, evaporation still occurs because the increase in air temperature between the air inlet and outlet increases the water-holding capacity of the air (Mitchell et li., 1999).

The desiccation of the substrate can lead to an unfavorably low water activity resulting in a poor microbial activity and subsequent channeling in the bed, (Weber et al., 2002). Using cooling water in the jacket of a small diameter packed bed bioreactor in order to increase the conduction effect is another way to control an operation in SSF (Saucedo Castaneda et al., 1990; Shojaosadati et al., 2007). However, for a large-scale process, it is not practical to use small cylindrical diameter beds.

In rectangular Zymotis large scale packed-bed bioreactor, the heat removal by radial conduction is promoted by the insertion of the closely-spaced internal heat transfer plates into the bed (Roussos et al., 1993). This reactor could be considered as a tray reactor where the layers of the substrates would be set vertically. Relatively little quantitative data is reported for the Zymotis bioreactor but enzyme levels are comparable to those obtained with the column bioreactors of 20 cm height and 2.2 cm diameter (Roussos et al., 1993). The Roussos's design of Zymotis packed-bed bioreactor contained a rectangular acrylic box which is fitted by acrylic dome-shaped cover and ten parallel stainless steel heat exchanger plates are placed inside the acrylic box. The bioreactor is sterilized with 70% ethanol and it is difficult to work in aseptic conditions. Another deficiency of the Zymotis design is a product harvesting method. At the end of fermentation, fermented substrate is collected by removing the internal plates under non-septic condition.

A theoretical method of analyzing the performance of Zymotis solid-state bioreactor based on productivity is demonstrated and the optimal cooling plate spacing and thickness using some given parameter values and operational conditions is simulated by Mitchell et al., 2000 and 2002. As mentioned above, there is little data on Roussos's Zymotis bioreactor design and also cultivation conditions. The variables are not monitored properly and product harvesting is carried out under non-septic condition. Hence there is need to contract a bioreactor for solid state fermentation overcoming above drawback.

### OBJECTIVE OF THE EMBODIEMNTS

The primary object of the embodiments herein is to provide a modified Zymotic bioreactor for solid state fermentation.

Another object of the embodiments herein is to provide aseptic method to cultivate microorganisms and harvest fermented products.

Yet another object of the embodiments herein is to provide moveable cooling plates having internal baffles for temperature control in bioreactor.

Yet another object of the embodiments herein is to produce the desired fermented product in a controlled environment and which is closely monitored using on-line sensors.

Further object of the embodiments herein is sterilizing the bioreactor and substrate by using steam at two stages.

### SUMMARY

The embodiments herein provide an improved Zymotis solid-state bioreactor and method for cultivation of microorganisms aseptically. Some aspects of preference of the modified Zymotis bioreactor and its operation are as follows. A modified Zymotis bioreactor with two compartments based on simulated data (Mitchell et al., 2000 and 2002) is constructed. The material and thickness of the construction material could be similar to a large scale bioreactor. The bioreactor is easy to be sterilized and handled under aseptic conditions. In one of the embodiments, a bioreactor with two compartments containing three cooling plates is constructed. These plates have internal baffles instead of cooling tubes. The bioreactor has temperature sensors loaded at different heights of the outer cooling plates for recoding and monitoring the bed temperature during a fermentation process. A short space is provided between the two cooling plates with a suitable material construction to permit the metabolic heat removal by a conduction process. The distance between the two compartments is adjusted manually and the best width of each compartment is investigated. The extraction and harvesting of the product can be carried out in place of the bioreactor using a trickle solvent. Productivity of this bioreactor is higher than that of one liter laboratory scale packed bed bioreactor. All of the on-line data are monitored on screen and recorded in the computer.

### BRIEF DESCRIPTION OF THE DRAWINGS

The embodiments herein will be better understood from the following detailed description with reference to the drawings, in which:

FIG. 1 illustrates a perspective view of the Zymotis solid state bioreactor, according to one embodiment of the present invention.

FIG. 2 illustrates a front side cross-sectional view of the Zymotis bioreactor to show cooling plates and compartments.

FIG. 3 illustrates a functional block diagram of the air supplying and controlling and on-line monitoring systems in Zymotis bioreactor according to one embodiment of the present invention.

### DETAILED DESCRIPTION OF THE EMBODIEMNTS

The embodiments herein provide a modified Zymotis bioreactor for cultivating microorganisms on solid medium under specified conditions. The bioreactor is constructed such that it combines all the operations involved in carrying out solid state fermentation (SSF) which includes sterilizing the bioreactor; inoculating the cultivation media with the microorganisms; cultivating the microorganisms under specified conditions and harvesting biological products from the cultivated microorganism.

The modified zymotis bioreactor of the embodiments herein provides significant advantages over known and existing bioreactors. The methods for cultivating microorganisms on a moist solid substrate in the areas of containment, material handling, control of the cultivation process, harvesting the products of interest. Containment, as used herein, refers to both the contained and aseptic nature of the bioreactor. The modified bioreactor of the embodiments herein operates in a contained manner by protecting the contents of the bioreactor from contamination by the outside environment (operating in an aseptic manner) and by protecting the environment from the potentially harmful or pathogenic microorganisms growing within the bioreactor (operating in a contained manner). The contained nature of this bioreactor not only allows the entire process of solid state fermentation to be carried out in isolation from the outside environment but also provides the advantage of maintaining a sterile environment throughout the entire fermentation process. The contained nature of the bioreactor according to the embodiments herein further provides the ability to sterilize, inoculate and control the fermenting medium temperature and moisture content in situ. Furthermore, the extraction of a biological product from the fermenting medium can be achieved easily. Following extraction of the product, the reactor contents may be sterilized in situ. The reactor may then be cleaned and reused for the next fermentation cycle.

The bioreactor is sterilized prior to inoculation thereby destructing all microorganisms from within the bioreactor prior to the start of the fermentation process. Steam is used in two stages of the fermentation. The steam is used in an upstream stage for sterilization of empty bioreactor before loading an inoculated substrate. The steam is used at the end of fermentation before harvesting the fermented substrate.

The bioreactor is sterilized prior to inoculation to destroy all microorganisms from within the bioreactor prior to the start of the fermentation process. This method also protects the microorganism being transferred from a contaminated environment. Air coming out of the bioreactor might carry spores and is filtered before being let out. Similarly inlet air sent into the bioreactor is also filtered to prevent contamination. After the product has been harvested from the bioreactor, the reactor may be sterilized in situ.

### The Bioreactor

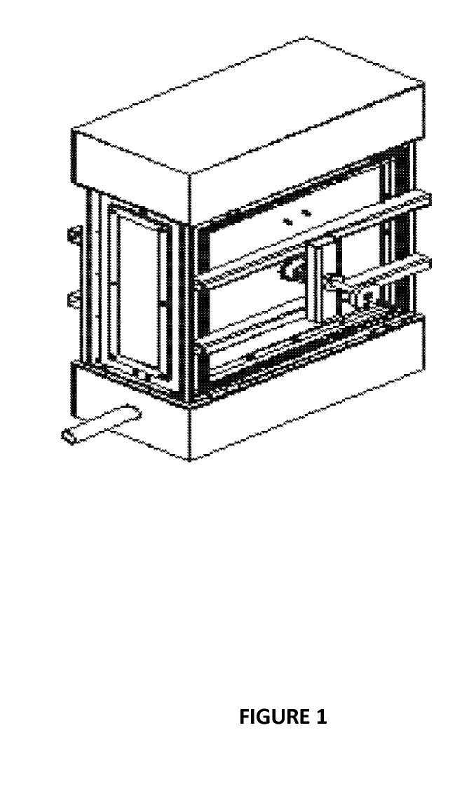

The modified Zymotis bioreactor of the embodiments herein is a rectangular packed bed bioreactor, with top and bottom equal height of headspace (FIG. 1). The size of a rectangular box in one of the embodiment is 0.26 m length×0.5 m width×0.76 m height (height of main body and two up and down boxes of the bioreactor). The outer casing of the bioreactor is insulated. Inside area of the bioreactor is partitioned by cooling plates arranged in parallel to each other. The numbers and dimensions of the cooling plates can be as large as is suitable for operation. In one embodiment, the dimension of each of cooling plate is of 0.012 m length×0.45 m width×0.5 m height. The bioreactor and cooling plates may be constructed by any suitable material that permits operation, sterilization and high conduction. The working capacity of the modified bioreactor 9 to 17 litter/compartment for 4 to 7.5 cm distance between each two cooling plates.

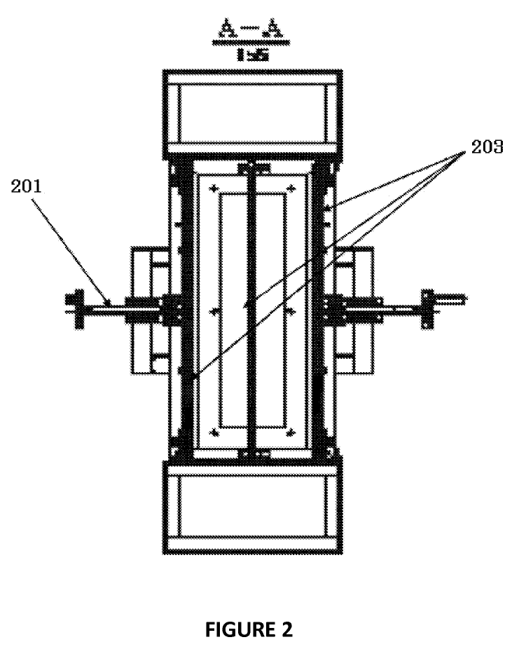

In the currently improved Zymotis bioreactor, two compartments of the bioreactor contain three cooling plates 202. Based on simulated data (Mitchell et al., 2000 and 2002), the bioreactor is constructed of stainless steel. The middle of the cooling plate is fixed and two other plates are movable (FIG. 2). FIG. 2 also shows handle for the bioreactor 201. So, it is possible to change distance between two plates and length of the beds to investigate its effect on efficiency and productivity of solid state fermentation. There are some baffles in each of the cooling plates instead of the cooling tubes and the plate is filled with water completely. The structure of the plate is simple and suitable for scaling up of a bioreactor. The inlet and outlet of water circulation points were located at the backside of each cooling plate.

A perforated plate is placed in an upper-side of the bottom headspace for supporting the bed and also sparging air. For extraction and recovery of the product, suitable solvent is trickled on the fermented cake from the perforated tubes located in the top headspace of the fermentor. The leachate is passed through the perforated plate and then collected from bottom headspace in an aseptic condition. The harvesting of the fermented solid is carried out easily by opening the bottom headspace and pushing out the cake of the fermented solids from the top to the bottom of each compartment.

### Bioreactor Operation

It is possible to sterilize the bioreactor using steam or sterilizing gas such as ethylene oxide. In the current operation, the bioreactor before loading is sterilized with steam and then inoculated sterilized substrate is loaded in the compartments up to a desired length. The various variables such as initial moisture content of solid substrate, nutrient and initial pH of substrate are investigated.

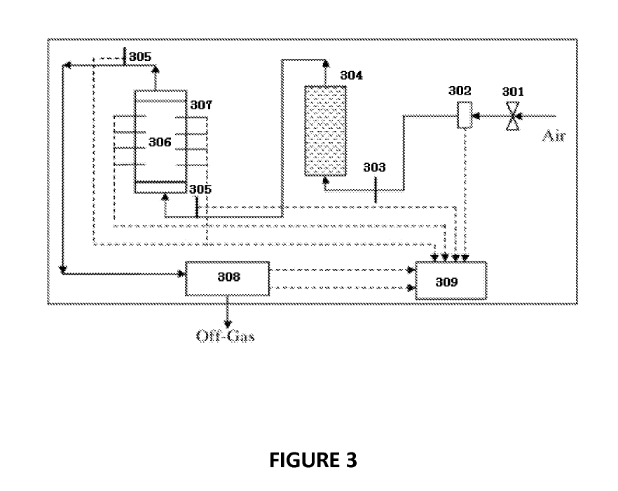

A schematic diagram of the fermentation process consisting of modified Zymotis packed bed bioreactor 306 with aeration 301, air filtration 303, humidifier system 304, control and on-line monitoring systems is shown in FIG. 3. The air flow rate 302, humidity and temperature 305 of inlet as well as outlet air and cooling water temperature in the interval plates are adjustable and controllable by sensors. Carbon dioxide production rate (CPR) 308 is measured as an indirect measurement of microbial growth. The bed temperatures are monitored by sensors 307 which placed in different heights of each compartment during fermentation. All of on-line data are monitored on screen and recorded by the computer 309.

Depending on the microorganisms and the aim of the operation, the product is harvested by leaching the fermented solid at the end of the fermentation and the bioreactor is evacuated easily by pushing out the cake of the fermented solids from the top to the bottom of each compartment.

### EXAMPLE 1

Several experiments with various initial moisture and pH of substrate, flow rates, cooling plate temperature and a certain distance between plates were investigated in the modified Zymotis packed bed bioreactor. One of the experiments is a simultaneous measurement of the bed temperature and moisture and microbial growth for Aspergillus niger, as a fast growing mold, on wheat bran in modified Zymotis packed bed bioreactor.

After sterilizing the bioreactor using steam, 8 kg of wet wheat bran was loaded in the bioreactor with suitable width of each compartment. The sterilized substrate inoculated with spore before loading in the sterile bioreactor. The spore suspension concentration is 5×10⁷ spore/ml and the concentration of spore in inoculated substrate was 5×10⁶ spore/(g initial dry weight substrate). The inoculated wheat bran was incubated for 3 days at a cooling plate temperature of 31° C. and an air flow rate of 14 liter per minute.

During this period, the bed temperature was controlled and maximum central bed temperature, axial and radial gradient temperatures were 38.6, 0.4 and 2.7 ° C. respectively. The Axial bed moisture gradient was low and it depends on the initial substrate moisture and air flow rate. In one embodiment, the maximum axial bed moisture gradient is about 18 (% w/w). SSF growth condition was suitable because of the high radial heat transfer conduction to the cooling plates. The Maximum productivity (4.3 g biomass/h. kg initial dry weight substrate) in 55 h fermentation time in the modified Zymotis is higher than that of a one liter lab scale packed bed solid state bioreactor achieved in the previous study (Shojaosadati et al. 2007) and in the article (Saucedo-Castaneda, et al. 1990).

While a presently preferred embodiment of the invention has been described with particularity, variation from the illustrated embodiment is possible without departure from the scope of the invention. This scope is to be determined by reference to the appended claims.

1. A modified zymotis packed bed bioreactor for cultivating microorganisms comprising:
a rectangular compartment comprising at least two cooling plates, wherein cooling pates are movable;
said cooling plates comprising at least one baffle located inside;
a means for adjusting a distance between said moveable cooling plates;
at least one sensor, wherein said at least one sensor is passed through said first cooling plate and said second cooling plate;
a means for sterilizing said bioreactor;
a means for aerating said bioreactor to obtain a predetermined amount of fermented solid substrate;
a means for extracting cellular products from said fermented solid substrate; and
a means for disposing said fermented solid substrate;
wherein microorganisms are cultivated on said solid substrate in such a manner that the bioreactor carries out all steps for cultivating microorganisms in an aseptic environment.

2. The modified zymotis bioreactor according to claim 1, wherein said two cooling plates are assembled vertically and parallel to each other.

3. The modified zymotis bioreactor according to claim 1, wherein said at least one sensor monitors said packed bed temperature.

4. The modified zymotis bioreactor according to claim 1, wherein said packed bed bioreactor is adapted to improve sterilization of said bioreactor.

5. The modified zymotis bioreactor according to claim 1, wherein said packed bed bioreactor is adapted to improve a heat transfer between said cooling plates.

6. The modified zymotis bioreactor according to claim 1, wherein said means for disposing disposes said fermented solid substrate from the bottom of said bioreactor by opening an aperture in the bottom of said bioreactor.

7. The modified zymotis bioreactor according to claim 1, wherein said sterilization occurs by steam, sterilizing gas or other suitable chemicals.

8. The modified zymotis bioreactor according to claim 1, wherein said means extracting cellular products from said fermented solid substrate trickles a solvent on said fermented solid substrate.
